(19) United States
(12) Patent Application Publication
(10) Pub. No.: US 2007/0140965 A1
(43) Pub. Date: Jun. 21, 2007

(54) Methods to ameliorate and image angioplasty-induced vascular injury

(75) Inventors: Gregory Lanza, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(73) Assignee: Barnes-Jewish Hospital, St. Louis, MO (US)

(21) Appl. No.: 11/607,683

(22) Filed: Dec. 1, 2006

Related U.S. Application Data
(60) Provisional application No. 60/741,929, filed on Dec. 2, 2005.

(51) Int. Cl. A61K 51/00; A61K 39/395; A61K 38/17; A61K 31/704; A61K 31/4745; A61K 31/337; A61K 31/336

(52) U.S. Cl. 424/1.49; 128/898; 514/2; 424/489; 514/291; 514/34; 514/449; 514/475; 514/731; 977/906

(57) ABSTRACT

Methods for inhibiting restenosis in blood vessels expanded by angioplasty are described. The method comprises administering blood vessel wall-targeted emulsion containing an anti-restenotic agent.

### RELATED APPLICATION

This application claims benefit of U.S. provisional application 60/741,929 filed 2 Dec. 2005. The contents of this document are incorporated herein by reference in their entirety.

### TECHNICAL FIELD

This invention relates to methods to prevent restenosis and ameliorate vascular injury induced by angioplasty. More particularly, the invention relates to the use of targeted particulate emulsions comprising a therapeutic agent that may aid in repair of an injured blood vessel, as well as retarding restenosis. Images of the injury may also be obtained.

### BACKGROUND ART

Clogged or constricted blood vessels are often treated by angioplasty—i.e., insertion of an inflatable device to effect opening of the vessel, usually followed by placement of a stent in the open vessel to address vascular recoil due to spasm or local wall dissections caused by the original procedure. The angioplasty procedure and the stents used have a number of drawbacks, including effecting neointimal proliferation leading to restenosis, and acute or delayed induction of arterial thrombosis, resulting in tissue ischemia or infarction. Although restenosis is a complication of percutaneous angioplasty, neointima development is increased and accelerated when stents, such as bare metal stents, are employed. Drug-eluting stents (DES) provide a scaffold benefit to address vascular spasm and dissection while also delivering local drugs which inhibit restenosis. Despite the benefits of drug eluting stents, these medical devices have several significant limitations. Due to size and inflexibility, many sites of stenosis in distal lesions or small branches cannot be treated with DES, resulting in the use of a traditional non-DES or no stent at all.

For lesions, where DES placement is feasible, the positioning of drugs to prevent restenosis along the lumen wall interface requires drug to diffuse into the wall where its positive effects are manifested. However, most of the drug never reaches its intramural target. The majority is washed from the stent and carried downstream, where it can impair the normal vasomotor functions of arterial wall, and the residual drug remaining with the stent, greatly delays intimal repair, a process dependent on the formation of the endothelial lining over the implanted metal struts of the stents. Because of this delay in healing, the risk of thrombosis is maintained for many months and up to two years. To counteract this complication, patients receive at least two and occasionally three anti-platelet drugs for six months to 2 years, with all the attendant risks and costs associated with anti-thrombosis therapy. Unfortunately, in some situations, despite all efforts to avoid these risks, the subject may experience acute episodes of ischemia, infarction and frequently death. In some cases these adverse events occur despite maximum medical therapy, in other instances treatment is prematurely discontinued by the physician or patient. In some situations patients must be withdrawn from these medicines to permit surgery or address bleeding complications, such as intracerebral hemorrhage following head trauma such as that sustained by a simple fall. In other cases, the cost of these medications exceeds patient financial resources over the multi-month or year(s). These realizations are discussed, for example, by Shuchman, M., New England J. of Med. (2006) 355:1949-1952. Reports have also appeared in the popular press to this effect.

Therefore, there is a need to devise treatments whereby anti-restenotic, anti-cell migratory, or anti-cell proliferative agents are targeted into the injured wall where the effect of these drugs is most effective and where the impact on intimal rehealing post angioplasty is least impacted, regardless of adjunctive stent placement.

PCT publication WO 2005/077407 describes emulsions of perfluorocarbon nanoparticles which contain antiproliferative agents for use in treating atherosclerosis and restenosis. Fumagillin is exemplified as an therapeutic antiangiogenic agent; rapamycin is an example of a drug with antirestenotic benefits. PCT publication WO 2003/062198 describes the use of emulsions targeting αᵥβ₃ in a restenosis model, wherein images are obtained by MRI. In both of these publications, it is suggested that these emulsions be administered well after angioplasty has already been conducted. Only systemic administration is described in these documents.

A description of MRI imaging of blood vessels in an angioplastic context has been published by the present inventors in an article by Cyrus, T., et al., J Cardiovasc. Magnet. Res. (2006) 8:535-541. Ultrasound imaging of stretch induced tissue factor expression in carotid arteries was described by Lanza, G., et al., Invest. Radiol. (2000) 35:227-234. Both documents employ porcine models. Neither describes delivery of therapeutic agents to the stretched vessel.

### DISCLOSURE OF THE INVENTION

The present invention may permit avoidance of stents and, in any case, will result in reduction in thrombosis and/or restenosis as a result of angioplasty by providing targeted emulsions containing anti-restenotic, anti-cell migratory, or anti-cell proliferative agent that allow the intima to heal and that prevent restenosis. These agents may be administered before or during the interventional procedure immediately following angioplasty. The emulsions may be targeted to epitopes on intramural cells, e.g., smooth muscle cells (SMC, or may be targeted to components of arterial extracellular matrix, e.g., collagen, contained in the vessel wall. Any accessible epitope(s) present in adequate concentration within the balloon-injured wall is satisfactory as a target. In general, endothelial cells lining the lumen are not targeted as they are typically destroyed and the vessel is denuded of intima by the angioplasty procedure itself. The targeted emulsions and their local delivery is designed to maximize distribution into the injured wall, and to minimize downstream losses where the composition is taken up into the blood flow of the primary or branch vessels.

Thus, in one aspect, the invention is directed to a method to ameliorate the restenosis resulting from angioplasty, which method comprises identifying a subject having a blood vessel that requires angioplasty, administering into said blood vessel, optionally at the location of the angioplasty, a targeted emulsion of particulates comprising an anti-restenotic, anti-cell migratory, or anti-cell proliferative agent optionally in combination with an ancillary imaging agent for imaging the vessel, and performing angioplasty of the blood vessel. The administering step is performed either before the angioplasty is conducted or concomitantly therewith. By “concomitantly therewith” is meant as a part of a single medical treatment; the administering of the emulsion may occur after the expansion of the vessel, but during the course of the same procedure. Typically, in this case, the administration will be local to the treated portion of the vessel.

If performed prior to the angioplasty, the administering may be done on a systemic basis. However, in all other embodiments, local administration is performed. It is often desirable to image the vessel as well. In some cases, imaging is possible using the particulate emulsion itself as a contrast agent. For example, many particulate emulsions are suitable contrast agents for ultrasound procedures, and, e.g., perfluorocarbon emulsions may behave as contrast agents for MRI by virtue of the presence of ¹⁹F. Emulsions with oils containing high atomic number atoms can serve directly as contrast agent in X-ray or other scanning procedures.

In another aspect, the invention is directed to a method to verify the delivery of therapeutic agent specifically to the blood vessel wall which method comprises obtaining an image of the blood vessel as described above.

### BRIEF DESCRIPTION OF THE DRAWINGS

FIG. 1 is a graph showing that the rate of endothelial healing is not affected by the presence of intramural targeted rapamycin nanoparticles as compared to saline after balloon-overstretch injury, i.e. angioplasty.

FIG. 2 is a graph showing that intramural targeted rapamycin emulsion particles reduce the neointimal plaque area of the vessel wall after balloon-overstretch injury, i.e. angioplasty.

FIG. 3 is a graph showing the distribution of percentage of stenosis calculated microscopically from approximately 6000 slides obtained from segments of blood vessels which were balloon-injured and treated with intramural targeted rapamycin emulsion (rapamycin), intramural targeted emulsion alone (no drug) or saline.

FIGS. 4a, 4b and 4c are images of balloon overstretch blood vessels in a pig. FIG. 4a is a time-of-flight angiogram; FIGS. 4b and 4c are MRI images using αᵥβ₃-targeted or collagen III-targeted nanoparticles as compared to the contralateral control.

FIGS. 5a, 5b and 5c are graphical representations of contrast-to-noise ratio, lesion length, and injury volume, respectively, as shown in images taken with control, αᵥβ₃-targeted and collagen III-targeted nanoparticle emulsions.

### MODES OF CARRYING OUT THE INVENTION

Blood vessels are composed of several annular layers. The lumen is formed by the intima which is comprised of vascular endothelial cells that are not proliferating and do not display integrin. In any event, the balloon-stretch injury, i.e., angioplasty, itself mechanically destroys most of the endothelial cells which must ultimately be replaced. The medial layer that surrounds the intima is composed of smooth muscle cells or myocytes which, when stimulated, proliferate and express tissue factor and integrins such as αᵥβ₃-integrin. This occurs when invasive procedures are performed. The medial layer is surrounded by the adventitial layer where neovascularization occurs and plaque formation is promoted. Surrounding the many cells of the blood vessel wall is an extracellular matrix comprised of various substances, including proteoglycans, collagens, fibronectin, tenacin, and vitronectin to name a few.

Restenosis is defined as the narrowing of a blood vessel, in particular, of an artery, that has been widened by a mechanical procedure, typically balloon-overstretch injury, commonly referred to as angioplasty. Frequently, stretch injury of a diseased vessel results in vascular spasm or vessel wall recoil, or dissections along the length of the vessels, which are countered with endovascular scaffold placement, i.e., stents. Although clinically significant neointimal proliferation is a known complication of angioplasty, placement of nondrug eluting stents typically worsens the severity and frequency of this adverse event. Thus, the presence of a stent to support the vessel wall usually amplifies the ingrowth of additional neointima, leading to restenosis. To ameliorate this to some extent, it is common to include drugs on the stent struts which diffuse from the lumen-wall interface into the deeper reached of the vessel wall. It is important that the drugs administered to prevent arterial restenosis do not inhibit the healing of the endothelial lining of the vessel, which is required to prevent intravascular thrombus formation at the injured site.

In one embodiment of the method of the invention, a catheter is used to deposit the targeted emulsion locally at the correct location in the blood vessel. The catheter should be designed to minimize the tendency of the emulsion to be lost downstream or via side branches of the blood vessel into the general circulation. Since regions of ruptured plaques or high grade stenosis are rich in intramural neovasculature, the emulsion bearing antirestenotic drugs may be administered systemically and targeted to angiogenic endothelial biomarkers, such as αᵥβ₃ integrin.

If the emulsion is administered prior to angioplasty, it may be administered immediately prior thereto, or may be administered an hour or less before the first balloon-inflation begins. For systemic administration, some lead-time is required before angioplasty is performed. For a local administration, however, the emulsion may be delivered immediately after the balloon expansion, which may then be optionally followed by stent placement. The stents used may be metal or polymeric, but would not incorporate antirestenotic drugs. Optionally, the implanted stent may incorporate drugs or growth factors, which would promote reendothelialization. Modifications of the angioplasty or similar balloon or a stent, permanent or erodible, to release the emulsion particles directly into the wall are also envisioned.

If the targeted emulsion is administered concomitantly with the angioplasty, a dual balloon or other local delivery catheter may be used. The dual balloon catheter is inserted and guided to the site of the blood vessel where angioplasty is needed. The two balloons are positioned at the site of angioplasty and the dispensing portion is located between the two balloons which are spaced longitudinally from each other by a few millimeters. The emulsion is then supplied locally in the space between the two balloons. It is thus delivered specifically to the interior of the blood vessel prior to, along with, or immediately after expansion of the balloons to effect the angioplasty. Modifications of this method are also within the scope of the invention wherein more than two balloons or multiple outlet ports are employed. Three, four or a multiplicity of balloons spaced appropriately apart similarly with areas for delivery of the emulsion may be used.

In another alternative, a 3-balloon catheter might comprise a single balloon for expansion and a dual balloon portion for administration of the emulsion. In this instance, the single balloon might first be inflated to expand the vessel, and the catheter then moved so that the space between the remaining two balloons, containing the emulsion to be administered, resides at the expanded portion of the vessel. The emulsion is then administered to the already expanded vessel.

Thus, in all cases, such “concomitant” administration occurs during the course of the same medical procedure, although the sequence of expansion and administering emulsion may be varied.

The amount of emulsion delivered will depend on the condition and nature of the subject, and on the judgment of the attending practitioner.

A stent may then be put into place, if desired.

Thus, by employing a targeted composition containing an suitable therapeutic agent to the interior of the blood vessel wall, the method of the invention is able to maximize the delivery of the emulsion to the affected area and minimize loss. Even if the emulsion is administered systemically prior to angioplasty, the neovasculature associated with the plaque requiring the treatment expanding the blood vessel may retain sufficient emulsion to be effective. The compositions of the invention do not interfere with repair of the intima, and are harmless in other areas of the blood vessel.

In addition to delivering the therapeutic agent, the compositions permit imaging of the surrounding tissue, if desired, so that verification can be obtained that the therapy has been delivered to the intended target.

In some cases the particles contained in the emulsion may themselves provide the imaging agent. If MRI is employed, typically the particles in the emulsion are provided with a chelating agent to associate a paramagnetic metal, although in some cases fluorocarbon particles may be used and the fluorine provides the contrast agent for MRI. If X-ray imaging is employed, a metal particle such as platinum or gold may be included. Radionuclides may also be included to provide imaging ability. Any suitable contrast agent may provide the basis for imaging, and such procedures are well known to the skilled artisan.

The targeting agents are designed to be bound by any component that is exposed in the interior of the wall of the blood vessel. Epitopes for binding the targeting agent will be those found on the surfaces of cells contained in the medial layer or vasa vasorum, such as tissue factor, integrins, other adhesion molecules, or receptors unique to these cells. The targeting agent may also bind specifically to components of the extracellular matrix including collagen and fibronectins. Any epitope that is present and exposed in the wall of the blood vessel provides a suitable target. Thus, suitable targeting agents are those that will bind these exposed epitopes and maximize the retention of the drug-containing particles of the emulsion at the desired location. The targeting agents themselves may be antibodies, aptamers, small molecules, peptidomimetics and the like—any moiety that has the capacity specifically to bind the desired target epitope.

The subjects to which the methods of the invention are applicable are most commonly humans, but also include any subject for which angioplasty can be practiced. Thus, the subjects may include household pets, sports animals, such as horses and dogs, farm animals, such as cows, pigs and sheep, including chickens and turkeys, and laboratory models for studying restenosis, such as rabbits, rats, mice, and the like.

### COMPOSITIONS OF THE INVENTION

A. Therapeutic Compounds

The emulsions used in the invention contain at least one therapeutic agent that prevents or ameliorates restenosis and does not interfere with the repair of the endothelial layer of a blood vessel expanded by angioplasty. The therapeutic agents are anti-restenotic, anti-proliferation and/or anti-cell migratory. More than one agent may be present in the emulsion.

The therapeutic agent may be supplied as a prodrug, including prodrug formulations as described, for example, by Sinkyla, et al., J. Pharm. Sci. (1975) 64:181-210, Koning, et al., Br. J. Cancer (1999) 80:1718-1725, U.S. Pat. Nos. 6,090,800 and 6,028,066 or as a conjugate, such as in PEGylated form.

Suitable therapeutic agents include, but are not limited to, matrix metalloproteinase (MMPs) inhibitors (e.g., inhibitors of MMP-2, MMP-9), tissue inhibitor of metalloproteinases (TIMPs, e.g., TIMP-1, TIMP-2, TIMP-3), marimastat, neovastat, thrombospondin-1, internal fragments of thrombospondin-1, METH-1 and METH-2 (proteins containing metalloprotease and thrombospondin domains and disintegrin domains in amino termini), fumagillin, fumagillin analogue TNP-470, endostatin, simvastatin, vasculostatin, vasostatin, angiostatin, protein kinase C beta inhibitor, genistein, anti-integrins, vascular endothelial growth factor inhibitor (VEGF-inhibitor), fragment of platelet factor-4 (amino-terminal fragment), derivative of prolactin, restin, angiopoietin-2 (antagonist of angiopoietin-1), proliferin-related protein, heparinase, antithrombin III fragment (fragment missing the carboxy-terminal loop of antithrombin III), bFGF-binding molecules, bFGF inhibitors, prolactin 16-kD fragment (derivative of prolactin), SPARC cleavage product, osteopontin cleavage product, thalidomide, squalamine, interferons (e.g., interferon-alpha, interferon-beta), interferon-inducible protein-10, anthracycline, 15-deoxyspergualin, D-penicillamine, eponemycin, herbimycin A, taxanes, such as paclitaxel and rapamycin. Radionuclides may also be used.

Therapeutic agents of use in the invention include agents which inhibit the activity of proangiogenic growth factors. Proangiogenic growth factor inhibitors may be in the form of antagonists which block or prevent effective production of a proangiogenic growth factor, antagonists which block or prevent effective binding of a proangiogenic growth factor to its receptor, and/or antagonists which block or prevent effective signaling of a proangiogenic growth factor. Agents with such inhibitor activity can be of a wide variety, including proteins (e.g., antibodies or antibody fragments), nucleic acids (e.g., antisense molecules, expression vectors encoding inhibitor), pharmaceuticals and the like. Examples of proangiogenic growth factors include vascular endothelial growth factor (VEGF), basic fibroblast growth factor (bFGF), acidic fibroblast growth factor (aFGF), fibroblast growth factor-3, fibroblast growth factor-4, transforming growth factor-alpha (TGF-alpha), epidermal growth factor (EGF), hepatocyte growth factor/scatter factor (HGF/SF), tumor necrosis factor-alpha (TNF-alpha), placental growth factor, platelet-derived growth factor (PDGF), granulocyte colony-stimulating factor, pleiotropin, interleukin-8, thymidine phosphorylase (TP)-platelet-derived endothelial cell growth factor (PD-ECGF), angiogenin and proliferin. Agents which may also inhibit VEGF activity include VEGF-neutralizing chimeric proteins such as soluble VEGF receptors and may be VEGF-receptor-IgG chimeric proteins. bFGF inhibitors may include bFGF-neutralizing chimeric proteins such as soluble bFGF receptors and may be bFGF-receptor-IgG chimeric proteins.

B. Targeting Ligands

The targeted carriers of the present invention employ targeting ligands for epitopes contained in the blood vessel wall. The targeting ligand serves to increase the concentration of the targeted carrier, and thus therapeutic agent, at a site of undesired angiogenic activity. As αᵥβ₃-integrin is expressed on SMC of the media, ligands specific for the αᵥβ₃-integrin can be used as targeting ligands in the present invention. As noted above, and in addition, other exposed epitopes at the surface of cells in the blood vessel, such as tissue factor, may be employed, as well as ligands that specifically target components of the extracellular matrix, such as fibronectins and collagens. The targeting ligands themselves may be antibodies or fragments thereof, peptidomimetics, aptamers, or even small molecules that serve as ligands for receptors. As used herein, “antibodies” includes both polyclonal and monoclonal antibodies, immunogenic fragments of the complete antibodies, recombinantly produced variants, such as Fₛᵥ single chain antibodies, and the like. Any antibody-related protein which displays the desired binding specificity is included in the definition of “antibody.”

C. Particulate Carriers

The emulsions themselves comprise particulates which can be of considerable variety. For example, PCT publication WO95/03829 describes oil emulsions where a drug is dispersed or solubilized inside an oil droplet and the oil droplet is targeted to a specific location by means of a ligand. U.S. Pat. No. 5,542,935 describes site-specific drug delivery using gas-filled perfluorocarbon microspheres. The drug delivery is accomplished by permitting the microspheres to home to the target and then effecting their rupture. Low boiling perfluoro compounds are used to form the particles so that the gas bubbles can form.

In another embodiment, nanoparticulate emulsions are based on high boiling perfluorocarbon liquids such as those described in U.S. Pat. No. 5,958,371. The nanoparticles are comprised of relatively high boiling perfluorocarbons surrounded by a coating which is composed of a lipid and/or surfactant. The surrounding coating is able to couple directly to a targeting ligand or can entrap an intermediate component which is covalently coupled to the targeting ligand, optionally through a linker, or may contain a non-specific coupling agent such as biotin. Alternatively, the coating may be cationic so that negatively charged targeting ligands such as nucleic acids, in general, or aptamers, in particular, can be adsorbed to the surface. The surface and/or core of the nanoparticulate emulsion also contains at least one therapeutic agent, e.g., an antiangiogenic agent, for delivery to the targeted cells or tissue. The outer coating thus provides for binding a multiplicity of copies of one or more desired components to the nanoparticle.

The nanoparticle emulsion and formulation for use in the methods of the invention, the construction of the basic particles and the formation of emulsions containing them, regardless of the components bound to the outer surface is described in U.S. Pat. Nos. 5,690,907, 5,780,010, 5,989,520 and 5,958,371, all of which are incorporated herein by reference.

Fluorocarbon emulsions and, in particular, perfluorocarbon emulsions are well suited for biomedical applications. The perfluorocarbon emulsions are known to be stable, biologically inert and readily metabolized, primarily by transpulmonic alveolae evaporation. Further, their small particle size easily accommodates transpulmonic passage and their circulatory half-life (“beta elimination” half time: 1-2 hours) advantageously exceeds that of other agents. Also, perfluorocarbons have been used to date in a wide variety of biomedical applications, including use as artificial blood substitutes. For use in the present invention, various fluorocarbon emulsions may be employed including those in which the fluorocarbon is a fluorocarbon-hydrocarbon, a perfluoroalkylated ether, polyether or crown ether. Useful perfluorocarbon emulsions are disclosed in U.S. Pat. Nos. 4,927,623, 5,077,036, 5,114,703, 5,171,755, 5,304,325, 5,350,571, 5,393,524, and 5,403,575 and include those in which the perfluorocarbon compound is perfluorotributylamine, perfluorodecalin, perfluorooctylbromide, perfluorodichlorooctane, perfluorodecane, perfluorotripropylamine, perfluorotrimethylcyclo-hexane or other perfluorocarbon compounds. Further, mixtures of such perfluorocarbon compounds may be incorporated.

As a specific example of a perfluorocarbon emulsion useful in the invention, a perfluorodichlorooctane or perfluorooctylbromide emulsion may include a lipid coating which contains between approximately 50 to 99.5 mole percent lecithin, preferably approximately 55 to 70 to mole percent lecithin, 0 to 50 mole percent cholesterol, preferably approximately 25 to 45 mole percent cholesterol and approximately 0.5 to 10 mole percent phosphatidylethanolamine, preferably approximately 1 to 5 mole percent phosphatidylethanolamine.

Lipid/surfactant coated nanoparticles are typically formed by microfluidizing a mixture of the oil or fluorocarbon which forms the core and the lipid/surfactant mixture which forms the outer layer in suspension in aqueous medium to form an emulsion. In this procedure, the lipid/surfactants may already be coupled to additional ligands when they are emulsified into the nanoparticles, or may simply contain reactive groups for subsequent coupling.

Alternatively, the components to be included in the lipid/surfactant layer may be solubilized in the layer by virtue of the solubility characteristics of the ancillary material. Sonication or other techniques may be required to obtain a suspension of the lipid/surfactant in the aqueous medium. Typically, at least one of the materials in the lipid/surfactant outer layer comprises a linker or functional group which is useful to bind the additional desired component or the component may already be coupled to the material at the time the emulsion is prepared.

The lipid/surfactants used to form an outer coating on the particles include natural or synthetic phospholipids, fatty acids, cholesterols, lysolipids, sphingomyelins, tocopherols, glucolipids, stearylamines, cardiolipins, plasmalogens, a lipid with ether or ester linked fatty acids, and polymerized lipids. In some instances, the lipid/surfactant can include lipid conjugated polyethylene glycol (PEG). Various commercial anionic, cationic, and nonionic surfactants can also be employed, including Tweens®, Spans®, Tritons®, and the like. In some embodiments, preferred surfactants are phospholipids.

Fluorinated surfactants which are soluble in the oil to be emulsified can also be used. Suitable fluorochemical surfactants include perfluorinated alkanoic acids such as perfluorohexanoic and perfluorooctanoic acids and amidoamine derivatives. Perfluorinated alcohol phosphate esters include the free acids of the diethanolamine salts of perfluoroalkyl phosphates.

Targeted particles in the emulsion may also be liposomes or niosomes. Liposomes may be prepared as generally described in the literature (see, for example, Kimelberg, et al., CRC Crit. Rev. Toxicol. (1978) 6:25; Yatvin, et al., Medical Physics (1982) 9:149; Lasic (1993) “Liposomes: from Physics to Applications” Elsevier, Amsterdam) and generally comprise lipid and amphipathic materials such as lecithin, sterols, egg phosphatidyl choline, and/or egg phosphatidic acid.

Liposomes are small vesicles composed of an aqueous medium surrounded by lipids arranged in spherical bilayers. Liposomes are usually classified as small unilamellar vesicles (SUV), large unilamellar vesicles (LUV), or multi-lamellar vesicles (MLV). SUVs and LUVs, by definition, have only one lipid bilayer, whereas MLVs contain many concentric bilayers. Liposomes may be used to encapsulate various therapeutic agents and materials, by trapping hydrophilic molecules in the aqueous interior or between bilayers, or by trapping hydrophobic molecules within the bilayer.

In some liposome embodiments, phospholipids are included and the liposomes may carry a net positive charge, a net negative charge or can be neutral. Inclusion of diacetyl-phosphate is a convenient method for conferring negative charge; stearylamine can be used to provide a positive charge. In some instances, at least one head group of the phospholipids is a phosphocholine, a phosphoethanolamine, a phosphoglycerol, a phosphoserine, or a phosphoinositol.

In some embodiments, the targeted particle is a lipid micelle or a lipoprotein micelle. Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins, et al., Int. J Pharm. (2000) 200:27-39. Lipoprotein micelles can be prepared from natural or artificial lipoproteins including low and high-density lipoproteins and chylomicrons.

In some embodiments, the targeted particle is a nanoparticle or microparticle which comprises a polymeric shell (nanocapsule), a polymer matrix (nanosphere) or a block copolymer, which may be cross-linked or else surrounded by a lipid layer or bilayer. Such lipid encapsulated nanoparticles and microparticles further comprise a therapeutic agent within the shell, dispersed throughout the matrix and/or within a hydrophobic core. General methods of preparing such nanoparticles and microparticles are described in the art, for example, in Soppimath, et al., J Control Release (2001) 70:1-20 and Allen, et al., J Control Release (2000) 63:275-286. For example, polymers such as polycaprolactone and poly(D,L-lactide) may be used while the lipid layer is composed of a mixture of lipid as described herein. Derivatized single chain polymers are polymers adapted for covalent linkage of a biologically active agent to form a polymer-agent conjugate. Numerous polymers have been proposed for synthesis of polymer-agent conjugates including polyamino acids, polysaccharides such as dextrin or dextran, and synthetic polymers such as N-(2-hydroxypropyl)methacrylamide (HPMA) copolymer. Suitable methods of preparation are described in the art, for example, in Veronese, et al., IL Farmaco (1999) 54:497-516. Other suitable polymers can be any known in the art of pharmaceuticals and include, but are not limited to, naturally-occurring polymers such as hydroxyethyl starch, proteins, glycopeptides and lipids. The synthetic polymers can also be linear or branched, substituted or unsubstituted, homopolymeric, co-polymers, or block co-polymers of two or more different synthetic monomers.

Other particulate-based emulsions can be formed from oil particles that contain components with high atomic numbers. These compositions are particularly useful as contrast agents in and of themselves. These are described in detail in PCT publication WO 2005/014051.

D. Ancillary Agents

In addition to the targeting ligand and therapeutic agent, the targeted carriers may contain or have associated with their surface an “ancillary agent” useful in imaging such as a radionuclide, a contrast agent for magnetic resonance imaging (MRI) or an agent for X-ray imaging or a fluorophore. The targeted carrier complexes themselves, in some instances can serve as contrast agents.

For example, radionuclides may be either therapeutic or diagnostic; diagnostic imaging using such nuclides is well known and by targeting radionuclides to desired tissue a therapeutic benefit may be realized as well. Radionuclides for diagnostic imaging often include gamma emitters (e.g., ⁹⁶Tc) and radionuclides for therapeutic purposes often include alpha emitters (e.g., ²²⁵Ac) and beta emitters (e.g., ⁹⁰Y). Typical diagnostic radionuclides include ^{99m}Tc, ⁹⁶Tc, ⁹⁵Tc, ¹¹¹In, ⁶²Cu, ⁶⁴Cu, ⁶⁷Ga, ⁶⁸Ga, and ¹⁹²Ir, and therapeutic nuclides include ²²⁵Ac, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹²³I, ¹³¹I, ⁶⁷Cu, ¹⁰⁵Rh, ¹¹¹Ag, and ¹⁹²Ir. The nuclide can be provided to a preformed particle in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the targeted carriers of the invention are understood in the art.

Chelating agents containing metal ions for use, for example, in magnetic resonance imaging can also be employed as ancillary agents. Typically, a chelating agent containing a paramagnetic metal or superparamagnetic metal is associated with the lipids/surfactants of the coating on the particles and incorporated into the initial mixture. The chelating agent can be coupled directly to one or more of components of the coating layer. Suitable chelating agents are macrocyclic or linear chelating agents and include a variety of multi-dentate compounds including EDTA, DPTA, DOTA, and the like. These chelating agents can be coupled directly to functional groups contained in, for example, phosphatidyl ethanolamine, oleates, or any other synthetic natural or functionalized lipid or lipid soluble compound. Alternatively, these chelating agents can coupled through linking groups.

Chelating agents appropriate for use in some instances include 1,4,7,10-tetraazacyclododecane-1,4,7,10-tetraacetic acid (DOTA) and its derivatives, in particular, a methoxybenzyl derivative (MEO-DOTA) and a methoxybenzyl derivative comprising an isothiocyanate functional group (MEO-DOTA-NCS) which can then be coupled to the amino group of phosphatidyl ethanolamine or to a peptide derivatized form thereof. Derivatives of this type are described in U.S. Pat. No. 5,573,752 and other suitable chelating agents are disclosed in U.S. Pat. No. 6,056,939.

The DOTA isocyanate derivative can also be coupled to the lipid/surfactant directly or through a spacer. The use of such spacers is described, for example, in PCT publication WO 2004/067483. The use of gly-gly-gly as a spacer is illustrated in the reaction scheme below. For direct coupling, the MEO-DOTA-NCS is simply reacted with phosphoethanolamine (PE) to obtain the coupled product. When a peptide is employed, for example a triglycyl link, PE is first coupled to t-boc protected triglycine. Standard coupling techniques, such as forming the activated ester of the free acid of the t-boc-triglycine using diisopropyl carbodiimide (or an equivalent thereof) with either N-hydroxy succinimide (NHS) or hydroxybenzotriazole (HBT) are employed and the t-boc-triglycine-PE is purified.

Treatment of the t-boc-triglycine-PE with trifluoroacetic acid yields triglycine-PE, which is then reacted with excess MEO-DOTA-NCS in DMF/CHCl₃ at 50° C. The final product is isolated by removing the solvent, followed by rinsing the remaining solid with excess water, to remove excess solvent and any un-reacted or hydrolyzed MEO-DOTA-NCS.

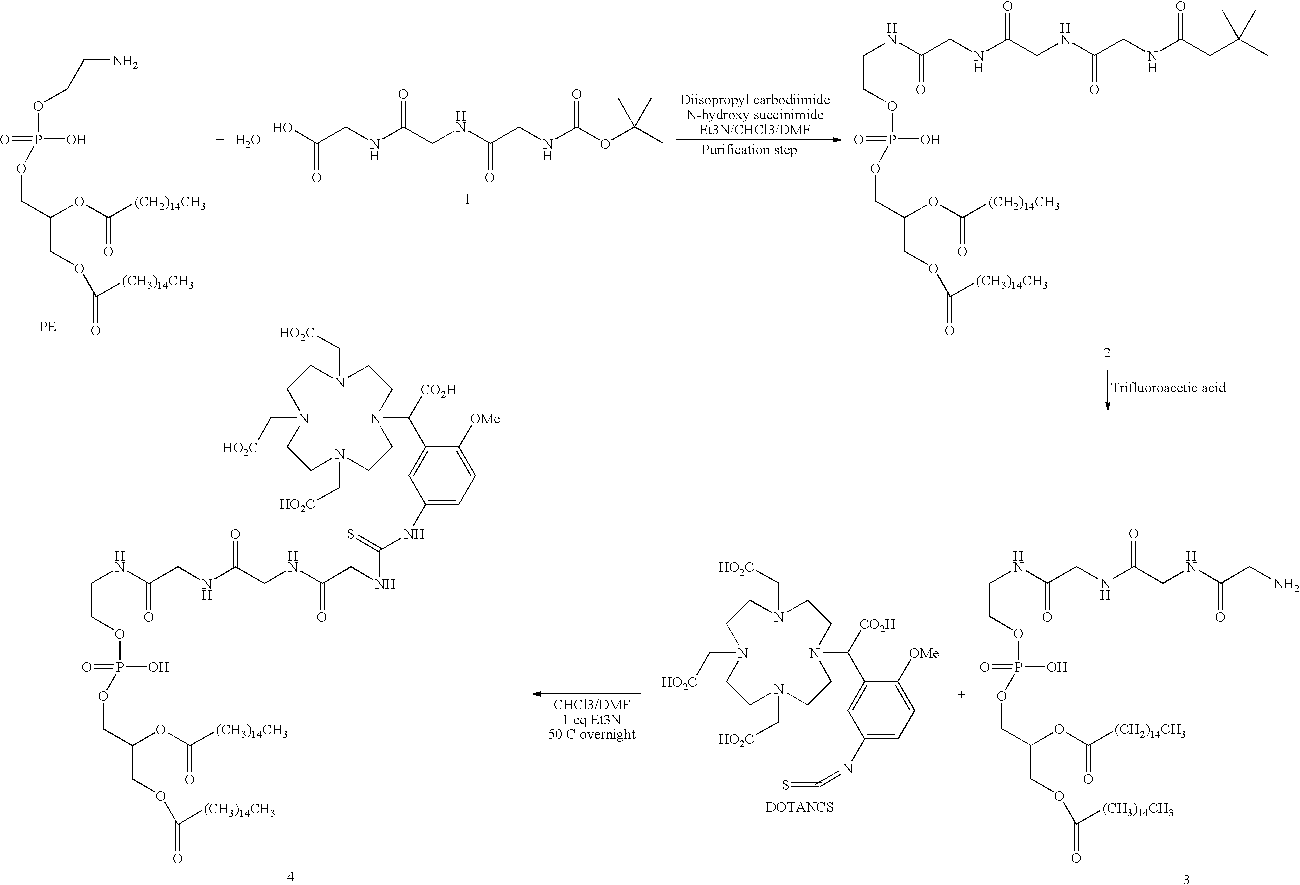

Other ancillary agents include fluorophores (such as fluorescein, dansyl, quantum dots, and the like) and infrared dyes or metals may be used in optical or light imaging (e.g., confocal microscopy and fluorescence imaging). For nuclear imaging, such as PET imaging, tosylated and ¹⁸F fluorinated compounds may be associated with the targeted carriers as ancillary agents.

E. Attachment of Components

The targeting ligands, drugs, and other components may be attached to the particulates in the emulsions in various ways. For example, in the case of nanoparticles which comprise lipid/surfactant coating layers, in some cases, included in the lipid/surfactant coating are components with reactive groups that can be used to couple the targeting ligand and/or the therapeutic agent and/or an ancillary substance useful for therapy and/or imaging. A lipid/surfactant coating which provides a vehicle for binding a multiplicity of copies of one or more desired components to the particle may be used. For example, phosphatidylethanolamine may be coupled through its amino group directly to a desired moiety, or may be coupled to a linker such as a short peptide which may provide carboxyl, amino, or sulfhydryl groups as described below. Alternatively, standard linking agents such a maleimides may be used. A variety of methods may be used to associate the targeting ligand, therapeutic agent and the ancillary substances, if any, to the particles; these strategies may include the use of spacer groups such as polyethylene glycol or peptides, for example.

Thus, the targeting ligand may be covalently bonded to a component of the lipid surfactant layer, such as phosphatidylethanolamine (PE), N-caproylamine-PE, n-dodecanylamine, phosphatidylthioethanol,N-1,2-diacyl-sn-glycero-3-phosphoethanolamine-N-[4-(p-maleimidophenyl) butyramide], 1,2-diacyl-sn-glycero-3-phosphoethanolamine-N-[4-(p-maleimidomethyl) cyclohexane-carboxylate], 1,2-diacyl-sn-glycero-3-phosphoethanolamine-N-[3-(2-pyridyldithio)propionate], 1,2-diacyl-sn-glycero-3-phosphoethanolamine-N[PDP(polyethylene glycol)2000], N-succinyl-PE, N-glutaryl-PE, N-dodecanyl-PE, N-biotinyl-PE, or N-caproyl-PE.

The covalent linking of the targeting ligands and/or other components to the materials in the lipid-encapsulated particles may be accomplished using synthetic organic techniques which would be readily apparent to one of ordinary skill in the art. The targeting or other ligand may be linked to the material, including the lipid, via the use of well known coupling or activation agents.

Typical methods for forming such coupling include formation of amides with the use of carbodiimides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. A multiplicity of ways to couple, covalently, a desired ligand to one or more components of the outer layer is well known in the art.

The ligand or other agent, including the therapeutic agent itself may be included in a surfactant layer if its properties are suitable. For example, if the ligand contains a highly lipophilic portion, it may itself be embedded in a hydrophobic coating. Further, if the targeting or other agent ligand and/or therapeutic agent is capable of direct adsorption to the coating, this too will effect its coupling. For example, nucleic acids, because of their negative charge, adsorb directly to cationic surfactants.

These noncovalent associations can also occur through ionic interactions involving a targeting ligand and/or therapeutic agent and residues within a moiety on the surface of the targeted particle. For example, noncovalent conjugation can occur between a generally negatively-charged targeting ligand or moiety on a nanoparticle surface and positively-charged amino acid residues, e.g., polylysine, polyarginine and polyhistidine residues. In another example, noncovalent conjugation can occur between a generally negatively-charged targeting ligand or moiety on an intermediate linker component and positively-charged amino acid residues of a therapeutic agent.

For example, the amino acid sequence Gly-Gly-His may be bound to the surface of an lipid-encapsulated nanoparticles covalently or by attachment to a hydrophobic moiety and copper, iron or vanadyl ion may then be added. Proteins, such as antibodies which contain histidine residues, may then bind to the lipid-encapsulated particles via an ionic bridge with the copper ion, as described in U.S. Pat. No. 5,466,467. Non-covalent associations can also occur through ionic interactions involving a targeting ligand and residues on a particle, such as charged amino acids.

As above, the ligand or other agent may bind directly to the particle, i.e., the ligand is associated with the particle or liposome itself. Binding may also be effected using a hydrolyzable anchor, such as a hydrolyzable lipid anchor, to couple the targeting ligand or other organic moiety to the lipid/surfactant coating of the particle. Indirect binding such as that effected through biotin/avidin may also be employed. For example, in biotin/avidin mediated targeting, the “targeting ligand” is coupled not to the particle or liposome, but rather coupled, in biotinylated form to the targeted tissue.

In this case, advantage is taken of avidin-biotin interactions. Avidin has a high affinity for biotin (10⁻¹⁵ M) facilitating rapid and stable binding under physiological conditions. “Biotin” includes biotin itself, as well as biocytin and other biotin derivatives and analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides and biotinylated amino acids such as N, epsilon-biotinyl-1-lysine. The term “avidin” includes avidin itself, streptavidin and other avidin analogs such as streptavidin or avidin conjugates, highly purified and fractionated species of avidin or streptavidin, and non-amino acid or partial-amino acid variants, recombinant or chemically synthesized avidin. Some targeted systems utilizing this approach are administered in two or three steps, depending on the formulation. Typically in these systems, a biotinylated ligand, such as a monoclonal antibody, is administered first and “pretargeted” to the molecular epitopes on the αᵥβ₃ integrin. Next, avidin is administered, which binds to the biotin moiety of the “pretargeted” ligand. Finally, the biotinylated emulsion is added and binds to the unoccupied biotin-binding sites remaining on the avidin thereby completing the ligand-avidin-emulsion “sandwich.” The avidin-biotin approach can avoid accelerated, premature clearance of targeted agents by the reticuloendothelial system secondary to the presence of surface antibody. Additionally, avidin, with four, independent biotin binding sites provides signal amplification and improves detection sensitivity.

Conjugations may be performed before or after an emulsion particle is created depending upon the compound to be conjugated. In a typical procedure for preparing nanoparticulate emulsions as targeted carriers of the invention, the core oil or oils and the components of the lipid/surfactant coating are fluidized in aqueous medium to form an emulsion. The functional components of the surface layer may be included in the original emulsion, or may later be covalently coupled to the surface layer subsequent to the formation of the nanoparticle emulsion. In one particular instance, for example, where a nucleic acid targeting agent or therapeutic agent is to be included, the coating may employ a cationic surfactant and the nucleic acid adsorbed to the surface after the particle is formed.

When appropriately prepared, the targeted carriers contain a multiplicity of functional such agents at their outer surface. For example, nanoparticles typically contain hundreds or thousands of molecules of the therapeutic agent, targeting ligand, radionuclide, and/or imaging contrast agent. For MRI contrast agents, the number of copies of a component to be coupled to the nanoparticle is typically in excess of 5,000 copies per particle, more preferably 10,000 copies per particle, still more preferably 30,000, and still more preferably 50,000-100,000 or more copies per particle. The number of targeting ligands per particle is typically less, of the order of several hundred while the concentration of PET contrast agents, fluorophores, radionuclides, and therapeutic agents is also variable.

In all of the foregoing cases, whether the associated moiety is a targeting ligand or therapeutic agent or an ancillary agent, the defined moiety may be non-covalently associated with the lipid/surfactant layer, may be directly coupled to the components of the lipid/surfactant layer, or may be indirectly coupled to said components through spacer moieties.

F. Emulsions

Generally, the emulsifying process involves directing high pressure streams of mixtures containing the aqueous solution, a primer material or the targeting ligand, the core oil or oils and a surfactant (if any) so that they impact one another to produce emulsions of narrow particle size and distribution. The MICROFLUIDIZER® apparatus (Microfluidics, Newton, Mass.) can be used to make the preferred emulsions. The apparatus is also useful to post-process emulsions made by sonication or other conventional methods. Feeding a stream of emulsion droplets through the MICROFLUIDIZER® apparatus yields formulations small size and narrow particle size distribution.

An alternative method for making the emulsions involves sonication of a mixture of oil(s) and an aqueous solution containing a suitable primer material and/or targeting ligand. Generally, these mixtures include a surfactant. Cooling the mixture being emulsified, minimizing the concentration of surfactant, and buffering with a saline buffer will typically maximize both retention of specific binding properties of the targeting ligand and the coupling capacity of the primer material. These techniques generally provide excellent emulsions with high activity per unit of absorbed primer material or targeting ligand.

The emulsion particle sizes can be controlled and varied by modification of the emulsification techniques and the chemical components. Techniques and equipment for determining particle sizes are known in the art and include, but not limited to, laser light scattering and an analyzer for determining laser light scattering by particles.

Emulsifying agents, for example surfactants, are used to facilitate the formation of emulsions and increase their stability. Typically, aqueous phase surfactants have been used to facilitate the formation of oil-in-water emulsions. Surfactant contain both hydrophilic and a hydrophobic portions.

Emulsifying and/or solubilizing agents may also be used. Such agents include, but are not limited to, acacia, diethanolamine, glyceryl monostearate, lanolin alcohols, lecithin, or mono- and di-glyceride.

As noted above, the emulsions can be prepared in a range of methods depending on the nature of the components. An illustrative procedure is as follows: perfluorooctylbromide (PFOB, 20% v/v), safflower oil (2% w/v), a surfactant co-mixture (2.0%, w/v), glycerin (1.7%, w/v) and water representing the balance is prepared. The surfactant co-mixture includes 58 mole % lecithin, 10 mole % cholesterol, 1.8 mole % phosphatidylethanolamine, 0.1 mole % peptidomimetic vitronectin antagonist conjugated to PEG₂₀₀₀-phosphatidylethanolamine (targeting ligand), and 30 mole % of gadolinium diethylene-triamine-pentaacetic acid-bis-oleate dissolved in chloroform. A therapeutic agent is added in titrated amounts between 0.01 and 50 mole % of the 2% surfactant layer, between 0.01 and 20 mole % of the 2% surfactant layer, between 0.01 and 10 mole % of the 2% surfactant layer, between 0.01 and 5.0 mole % of the 2% surfactant layer, preferably between 0.2 and 2.0 mole % of the 2% surfactant layer. The chloroform-lipid mixture is evaporated under reduced pressure, dried in a 50° C. vacuum oven overnight and dispersed into water by sonication. The suspension is transferred into a blender cup (for example, from Dynamics Corporation of America) with oil in distilled or deionized water and emulsified for 30 to 60 seconds. The emulsified mixture is transferred to a Microfluidics emulsifier and continuously processed at 20,000 PSI for four minutes. The completed emulsion is vialed, blanketed with nitrogen and sealed with stopper crimp seal until use. Control emulsions can be prepared identically excluding the therapeutic agent and/or the targeting ligand from the surfactant co-mixture. Particle sizes are generally determined in triplicate at 37° C. with a laser light scattering submicron particle size analyzer (Malvern Zetasizer 4, Malvern Instruments Ltd., Southborough, Mass.), which indicate tight and highly reproducible size distribution. Unincorporated therapeutic agent can be removed from the emulsion by dialysis or ultrafiltration techniques.

Kits

The targeted carriers of the invention may be prepared and used directly in the methods of the invention, or the components of the targeted carriers may be supplied in the form of kits. The kits may comprise the untargeted composition containing at least one therapeutic agent and all of the desired ancillary materials in buffer or in lyophilized form. The kits may comprise the pre-prepared targeted composition containing at least one therapeutic agent and all of the desired ancillary materials and targeting materials in buffer or in lyophilized form. Alternatively, the kits may include a form of the targeted carrier which lacks the targeting agent which is supplied separately or the kits may include a form of the targeted carrier which lacks the therapeutic agent which is supplied separately. The component(s) for the targeted carrier will contain a reactive group, such as a maleimide group, which, when the component is mixed with the targeting agent and/or therapeutic agent, effects the binding of the targeting agent and/or the therapeutic agent to the targeted carrier itself. A separate container may also provide additional reagents useful in effecting the coupling. Alternatively, the component(s) for the targeted carrier may contain reactive groups which bind to linkers coupled to the desired component(s) to be supplied separately which itself contains a reactive group. A wide variety of approaches to constructing an appropriate kit may be envisioned. Individual components which make up the ultimate targeted carrier may thus be supplied in separate containers, or the kit may simply contain reagents for combination with other materials which are provided separately from the kit itself.

A non-exhaustive list of combinations might include: targeted carrier preparations that contain, in their lipid-surfactant layer, the therapeutic agent and an ancillary component, if any, such as a fluorophore or chelating agent and reactive moieties for coupling to the targeting ligand; the converse where the targeted carrier is coupled to targeting ligand and optionally contains reactive groups for coupling to the therapeutic agent and to an ancillary material, if any; emulsions which contain both targeting ligand and therapeutic agent and possibly a chelating agent but wherein the metal to be chelated is either supplied in the kit or independently provided by the user; preparations of the nanoparticles comprising the surfactant/lipid layer where the materials in the lipid layer contain different reactive groups, one set of reactive groups for a targeted ligand, one set of reactive groups for a therapeutic agent and another set of reactive groups for an ancillary agent; preparation of targeted carriers containing any of the foregoing combinations where the reactive groups are supplied by a linking agent.

The following Examples are offered to illustrate but not to limit the invention.

### PREPARATION A

### Preparation of Emulsions

Emulsions of paramagnetic perfluorocarbon nanoparticles targeted to αᵥβ₃-integrins are prepared as described in Winter, et al., Circulation (2003) 108:2270-2274. In general, the nanoparticulate emulsions are comprised of 20% (v/v) perfluorooctylbromide (PFOB; Minnesota Manufacturing and Mining), 2% (w/v) safflower oil, 2% (w/v) of a surfactant co-mixture, 1.7% (w/v) glycerin and water for the balance. The surfactant co-mixture includes 58 mole % lecithin (Avanti Polar Lipids, Inc.), 10 mole % cholesterol (Sigma Chemical Co., St. Louis, Mo.), 0.1 mole % peptidomimetic vitronectin antagonist (U.S. Pat. No. 6,322,770) conjugated to PEG₂₀₀₀-phosphatidylethanolamine (Avanti Polar Lipids, Inc.), 1.8 mole % phosphatidylethanolamine (Avanti Polar Lipids, Inc.), and 30 mole % of gadolinium diethylene-triamine-pentaacetic acid-bis-oleate (Gd³⁺, Gateway Chemical Technologies) (U.S. Pat. No. 5,571,498).

Nanoparticulate formulations for use in local delivery of rapamycin include 0.2 mole % of rapamycin in the surfactant mixture at the proportionate expense of lecithin. Non-targeted nanoparticles exclude the integrin homing ligand, which is replaced in the surfactant mixture by an equivalent increase in phosphatidylethanolamine.

The surfactant components are prepared as described in Lanza, et al., Circulation (2002) 106:2842-2847 and in Winter, et al., Circulation (2003) 108:2270-2274, and combined with PFOB, safflower oil and distilled deionized water. The mixture is emulsified in a M110S Microfluidics emulsifier (Microfluidics, Inc, Newton, Mass.) at 20,000 PSI for four minutes. Particle sizes are determined at 37° C. with a laser light scattering submicron particle analyzer (Malvern Instruments, Malvern, Worcestershire, UK). The concentrations of Gd³⁺and nanoparticles in the emulsion are measured and the number of Gd³⁺complexes per nanoparticle is calculated.

Rapamycin nanoparticle emulsions (250 μl) are dialyzed in 60,000 MW cutoff dialysis tubing against 3.5 ml of releasing medium (0.9% NaCl, 0.2 mg/ml human serum albumin and 0.05% sodium azide) and continuously agitated at 37° C. The releasing medium is replaced daily and analyzed for released rapamycin concentration. Rapamycin is analyzed by reverse-phase HPLC (Waters Corporation). Chromatography is performed using a Waters Novapak C₁₈, 60 Å, 4 μm reversed-phase column (3.9×150 mm) with an isocratic 50% acetonitrile/0.05% of phosphoric acid mobile phase (1 ml/min at ambient temperature).

### EXAMPLE 1

Rabbits were fed an atherogenic diet for three weeks, and then subjected to balloon stretch injury. A catheter was inserted from the left common carotid artery, and a double balloon expanded into each artery. From the space between the two balloons, in test rabbits, αᵥβ₃ nanoparticles prepared as in preparation A and comprising rapamycin were administered. In a control contralateral vessel, the emulsion prepared in preparation A without rapamycin was administered.

The test and controlled arteries were imaged by MRI contrast enhancement to detect the injury pattern and the distribution of nanoparticles. Plaque development after treatment was determined by microscopic methods.

Imaging with the αᵥβ₃ integrin-targeted paramagnetic nanoparticles showed delineation of the stretch injury pattern. Magnetic resonance imaging is performed at 1.5 T, a clinically relevant field strength, using a clinical scanner (NT Intera CV, Philips Medical Systems) and a quadrature birdcage radiofrequency receive coil.

Two weeks after the injury, serial vascular sections were subjected to microscopic analysis. This showed that plaque increase in the vessels treated with targeted rapamycin was only about 12±1% whereas in vessels treated only by targeted nanoparticles without rapamycin the increase was 21±1.4%.

### EXAMPLE 2

New Zealand White Rabbits were fed 0.25% cholesterol diet for four months which resulted in plaque formation in the femoral artery. The artery was opened using balloon angioplasty using a dual balloon catheter and dispensing 0.4 ml of αᵥβ₃-integrin-targeted perfluorocarbon nanoparticles containing 0.3 mol % rapamycin in 12 of the rabbits, or non-targeted nanoparticles in 6 of the rabbits, or saline in 6 of the rabbits over the course of five minutes. The release of the drug was determined with dissolution studies and after 3 days more than 97% of the rapamycin was still incorporated in the nanoparticle emulsion. The emulsion also contained ^{99m}Tc label permitting detection of the local delivery of the targeted emulsion, but not the nontargeted emulsion into the femoral arteries.

Stenosis developed in balloon-injured, but untreated femoral arteries, but not in those exposed to the αᵥβ₃-targeted perfluorocarbon nanoparticles with rapamycin A, 4 weeks after injury.

Microscopic analysis of serial vascular sections revealed that the intimal plaque to lumen area ratio of the vessels treated with the targeted rapamycin nanoparticles was significantly less (14±1%) than in arteries receiving targeted nanoparticles without drug (21±1.4%) or saline (22±1.9%). In addition, rapamycin nanoparticle treatment led to smaller lesions than the two controls and no difference was observed in the rate of healing between the various treatments.

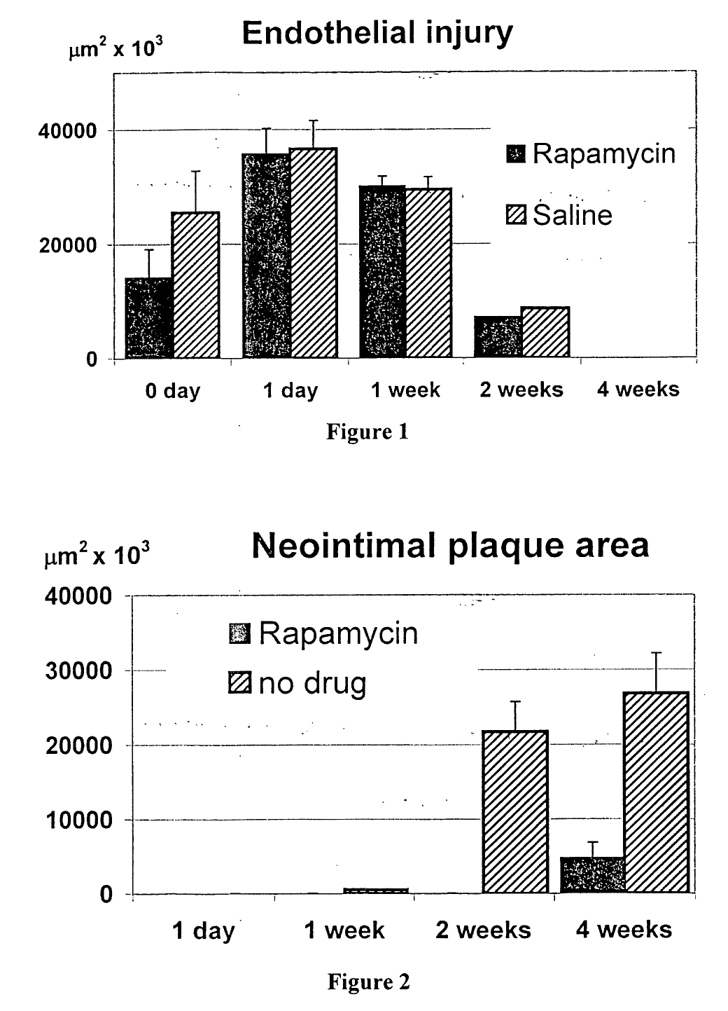

FIG. 1 shows that the extent of endothelial injury was not affected by the presence of rapamycin as compared simply to saline after the administration of the balloon expansion. The rapamycin-containing emulsion greatly reduced the neointimal plaque area, as shown in FIG. 2. In FIG. 1, the extent of endothelial injury is plotted in terms of area and is in the range of 20-40 μm². The plaque area shown in FIG. 2 is about 25 μm² for untreated subjects but only about 5 μm² in subjects treated with the rapamycin emulsion.

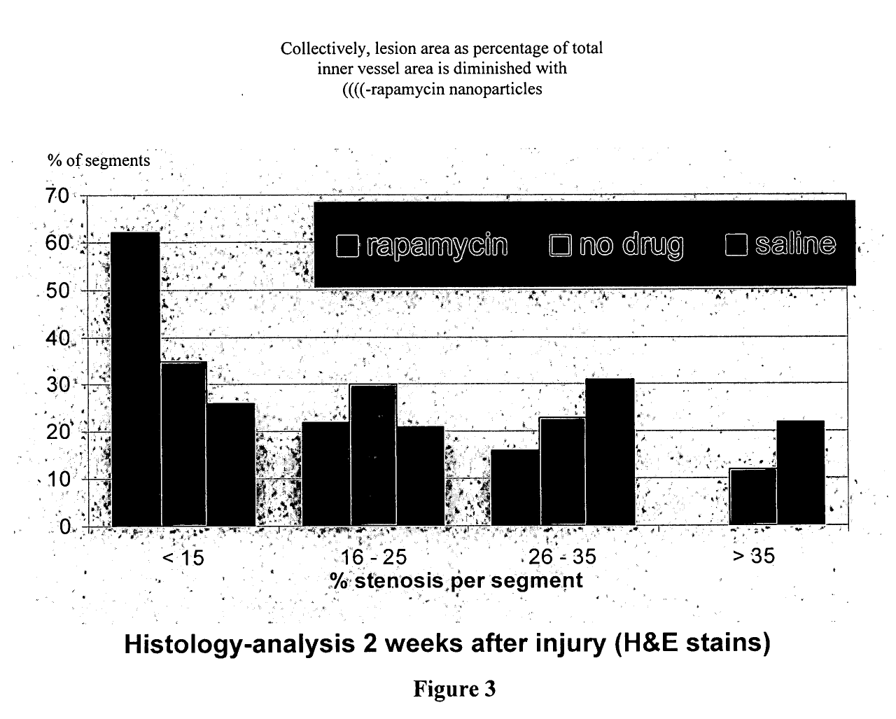

FIG. 3 shows that rapamycin-containing emulsions led to smaller lesions than did controls. To obtain the data in FIG. 3, multiple sections of the vessels were obtained, treated with hematoxylin and eosin (H&E) stain. Multiple sections from each vessel were examined. In FIG. 3, the percent stenosis in each segment was plotted as the X-axis and the number of segments exhibiting stenosis in the indicated range plotted on the Y-axis. As shown, the percentage of rapamycin-treated segments with <15% restenosis is more than 60%, whereas only 35% of segments from vessels treated with emulsions containing no drug and 25% of those treated with saline contained this small amount. For segments containing more than 35% stenosis, the rapamycin vessels yielded no segments with this high value, whereas for the saline control, for example, 20% of the segments fell into this range. Targeting agent in this case was directed to αᵥβ₃.

### EXAMPLE 3

Ligand-targeted paramagnetic nanoparticles were prepared as previously described. Briefly, the nanoparticles comprised 20% (volume/volume) perfluorooctylbromide (PFOB; Exfluor Research, Round Rock, Tex., USA) and 1.5% (weight/volume) of a surfactant co-mixture, 1.7% (w/v) glycerin and water for the balance. The surfactant co-mixture included 69.9 mole % lecithin (Avanti Polar Lipids, Inc., Alabaster, Ala., USA), 0.1 mole % peptidomimetic vitronectin antagonist (Bristol-Myers Squibb Medical Imaging, Billerica, Mass., USA) or anti-collagen III f_{(ab)} (CSIRO, Victoria, Australia) coupled to MPB-PEG₂₀₀₀-phosphatidylethanolamine (Northern Lipids, Inc., Vancouver, British Columbia, Canada), and 30 mole % of gadolinium diethylene-triamine-pentaacetic acid-bis-oleate (Gateway Chemical Technologies, St. Louis, Mo., USA). Nontargeted, paramagnetic particles were prepared by substituting the ligand-lipid conjugate with lecithin. The nominal sizes for each formulation were measured with a submicron particle analyzer (Malvern Zetasizer, Malvern Instruments, Malvern, Pa., USA) and were 245 nm±117 nm for the αᵥβ₃-targeted, 262 nm±99 nm for the collagen III-targeted, and 323 nm±26 nm non-targeted control nanoparticles.

All studies were approved by the Washington University Animal Studies Committee and are based on National Institutes of Health laboratory standards. Healthy domestic pigs weighing 20 kg were fed a normal diet (n=12). Animals were fasted overnight before sedation with telazol cocktail (1 mL/23 kg IM) followed by intubation and 1-2% isoflurane anesthesia in oxygen. The ECG, blood gases and arterial blood pressure were monitored. A 12F (size necessary to fit the double-balloon catheter during incubation) catheter sheath was aseptically inserted into the femoral artery via a cut-down and a bolus of heparin (200 U/kg) was given to inhibit clot formation in catheters. No antiplatelet agents were administered. A guide catheter was placed under fluoroscopy into the left or right carotid artery at the level of the 5th cervical vertebra. A baseline carotid angiogram was obtained and lidocaine and nitroglycerin were used to treat vasospasm. An 8 mm×2 cm balloon catheter (Proflex, Mallinckrodt Inc, St. Louis, Mo., USA) was positioned at the level of the 2nd and 3rd cervical vertebrae and inflated three times to a pressure of 6 atmospheres for 30 seconds with 60 second pauses between inflations. A balloon-to-artery ratio of approximately 1.5 was employed. This procedure produces a consistent rupture of the internal elastic lamina and injury to the media.

Following carotid overstretch-injury, nanoparticles were administered via a local delivery with a double-balloon catheter system (Edwards Lifesciences, Irvine, Calif., USA). The 7F double balloon catheter was inserted via the sheath in the right femoral artery and guided into the respective carotid artery. The inner distance between the distal and the proximal balloons was 6 cm. Under fluoroscopy, the catheter was placed in a fashion that the injured vessel segment was positioned in the middle between the two balloons. The site of injury had been marked both on X-ray and on the overlying skin during the injury. Upon satisfactory confirmation of the double-balloon catheter position, the proximal and then distal balloons were each gently (1 atm) inflated to occlude the artery. Blood was aspirated through the central porthole, and the arterial segment flushed with normal saline. Targeted nanoparticles (n=9 for αᵥβ₃-integrin and n=6 for collagen III) or non-targeted control nanoparticles (n=3; into the contralateral artery), or saline alone as control (n=6) were delivered locally and allowed to incubate for 10 minutes. The solutions were then withdrawn from the vessel and segment flushed thoroughly with saline before carotid flow was reestablished. A post-angioplasty carotid angiogram was obtained, and the animals were transferred for MR imaging of the neck vasculature.

Animals were imaged with MRI using a 1.5 Tesla clinical scanner (NT Intera CV, Philips Medical Systems, Cleveland, Animals were imaged with MRI using a 1.5 Tesla clinical scanner (NT Intera CV, Philips Medical Systems, Cleveland, Ohio, USA) and techniques optimized to assess persistence of contrast enhancement and in vivo luminal dimensions throughout the injured vessels. A 5-element phased array surface coil operating in the receive mode was used. Multislice T₁-weighted, gradient-echo, fat-suppressed, time-of-flight angiograms of the carotid arteries from the carotid origin to the bifurcation into external and internal carotid were performed with repetition times (TR) of 40 ms and echo times (TE) of 4.6 ms. T₁-weighted, fat-suppressed, fast spin-echo (TSE) imaging was performed to image the vascular wall (TR=532 ms, TE=11 ms, 250×250 μm in-plane, 2 mm slice thickness, echo train=4, number of signals averaged=8). To ensure complete nulling of the blood signal, “sliding” radiofrequency saturation bands were placed proximal and distal to the region of image acquisition and moved with the selected imaging plane. Contrast to noise between the nanoparticles and surrounding tissue was calculated as the difference of the signal between the nanoparticle targeted area and a region of interest within the surrounding tissue, respectively, divided by the standard deviation of the background signal. Contrast image analysis was performed with Easy Vision v5.1 (Philips Medical Systems, Cleveland, Ohio, USA) using regions of interest manually applied in each slice of the T₁-weighted baseline images. The segmented slices were reconstructed into a three-dimensional object to calculate the volume.

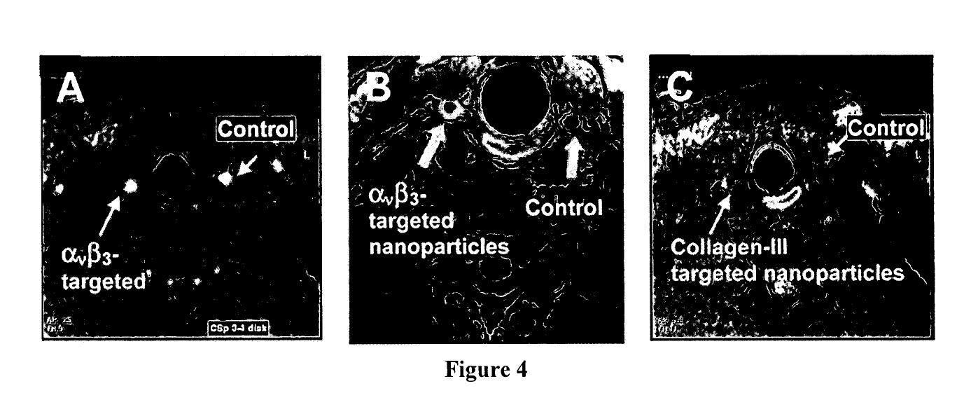

FIGS. 4a, 4b and 4c show the results of imaging. FIG. 4a is a time-of-flight angiogram depicting blood flow; FIG. 4b shows an image of the vessels either exposed to αᵥβ₃ integrin-targeted nanoparticles or non-targeted control particles and FIG. 4c shows a comparison of imaging obtained from collagen III targeted nanoparticles as compared to untargeted control.

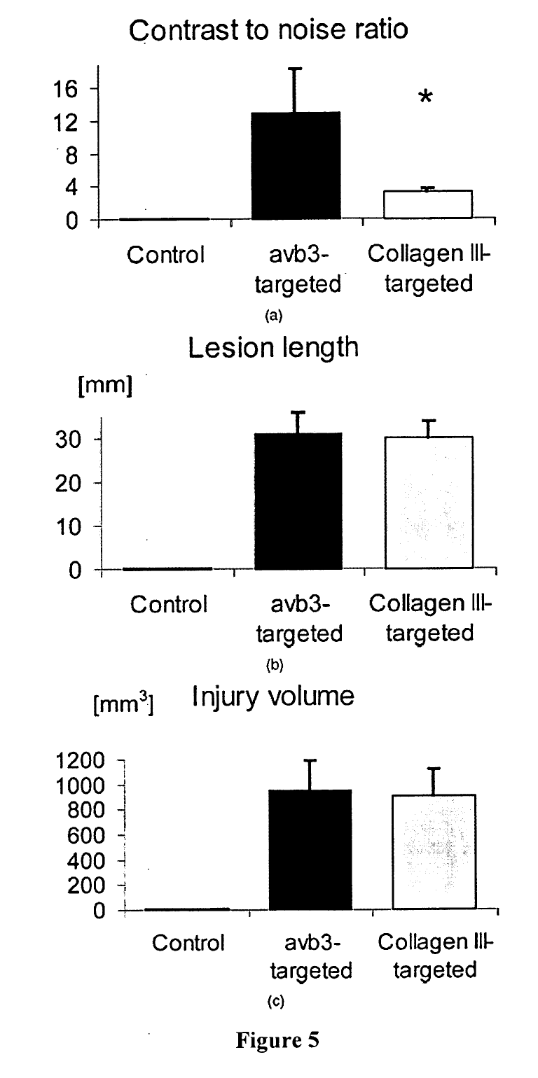

FIG. 5a, 5b and 5c show comparisons of contrast-to-noise ratio, lesion length, and injury volume, respectively, for vessels which are exposed to non-targeted emulsions (control) or to αᵥβ₃ or collagen III targeted emulsions.

As shown, only the targeted particles provided satisfactory imaging.

1. A method to inhibit restenosis associated with expansion by angioplasty in a blood vessel which method comprises
a) identifying a subject that comprises a blood vessel that will be subjected to angioplasty;
b) administering into said blood vessel, optionally at the location of the angioplasty, an emulsion of particulates wherein said particulates contain at least one targeting ligand specific for an exposed epitope in the blood vessel wall, at least one anti-restenotic, anti-cell migratory, or anti-cell proliferative agent, and optionally an ancillary imaging agent; and
c) subjecting said blood vessel to angioplasty;
wherein step b) is performed before step c), or during the same procedure in which step c) is performed.

2. The method of claim 1, wherein step b) comprises administering said emulsion at the location of the angioplasty.

3. The method of claim 1, wherein the targeting ligand maximizes delivery of the emulsion to the blood vessel wall at the location of the angioplasty and minimizes loss of said emulsion to the blood-flow in said blood vessel or to branches thereof.

4. The method of claim 3, wherein the exposed epitope is a component of the extracellular matrix or is displayed on a cell surface in said blood vessel wall.

5. The method of claim 4, wherein said exposed epitope is a component of the extracellular matrix.

6. The method of claim 5, wherein the exposed epitope is collagen or fibronectin.

7. The method of claim 4, wherein the exposed epitope is tissue factor, an integrin or other cell-surface moiety.

8. The method of claim 7, wherein the integrin is αᵥβ₃-integrin.

9. The method of claim 1, wherein the subject is a human, a household pet, or a laboratory animal.

10. The method of claim 1, wherein the particulates are nanoparticles comprising a perfluorocarbon core coated with a lipid/surfactant layer.

11. The method of claims 1, wherein the targeting agent is a peptidomimetic or an antibody.

12. The method of claim 1, wherein the at least one antirestenotic, antimigratory, or antiproliferative agent is rapamycin, doxorubicin, paclitaxel, probucol, PDGFRβ-specific tyrphostin, or fumagillin.

13. The method of claim 1, wherein the ancillary imaging agent comprises a chelate and a paramagnetic ion.

14. The method of claim 1, wherein the ancillary imaging agent comprises a radionuclide.

15. The method of claim 1, wherein the ancillary imaging agent comprises a heavy metal.

16. The method of claim 1, which further includes imaging the blood vessel at the site of angioplasty.
